(19) United States
(12) Patent Application Publication
(10) Pub. No.: US 2010/0310478 A1
(43) Pub. Date: Dec. 9, 2010

(54) SUBSTITUTED BENZOQUINONES AND HYDROQUINONES IN THE TREATMENT OF PERIODONTAL DISEASES

(76) Inventors: Daniel James Fitzgerald, Lees (GB); Nicholas Edward Read, Bradford (GB); Priscila Gottardello, Bradford (GB); Stephen Philip Jones, Woking (GB)

Correspondence Address: Weaver Austin Villeneuve & Sampson LLP, P.O. BOX 70250, OAKLAND, CA, 94612-0250, US

(21) Appl. No.: 12/747,480

(22) PCT Filed: Dec. 11, 2008
(86) PCT No.: PCT/GB08/04071

§ 371 Date: Aug. 25, 2010

(30) Foreign Application Priority Data
Dec. 13, 2007 (GB) 0724278.7

(51) Int. Cl. A61K 8/35; A61Q 11/00; C07C 49/782; C07C 49/543

(52) U.S. Cl. 424/49; 568/327; 568/377

(57) ABSTRACT

An alkyl- or halo-substituted benzoquinone, hydroquinone or mixture thereof, for use in the treatment of a periodontal disease, and/or for use in the treatment of a condition which is caused by, transmitted by or exacerbated by P. gingivalis or by biofilm formation by P. gingivalis.

### FIELD OF THE INVENTION

This invention relates to the use of certain types of quinone as antibacterial agents in the treatment of periodontal diseases and/or against bacteria of the species P. gingivalis.

### BACKGROUND TO THE INVENTION

Periodontal disease is a broad term used to describe a continuum of degenerative diseases which attack the tissues that surround and support the teeth (periodontium). A large majority of periodontal diseases are caused by bacteria that reside at or below the gum margin. The two commonest forms are gingivitis and periodontitis.

Gingivitis (inflammation of the gums or gingiva) typically results from poor dental hygiene. The oral cavity is colonised by over 500 species of bacteria which live in complex hierarchical communities at various sites in the mouth. Some of these bacteria are able to stick to tooth surfaces. When the teeth are not brushed properly, different species of bacteria that cannot stick to teeth directly, begin to stick to the early colonisers. This process begins to form the material described as dental plaque. Bacteria which comprise the plaque that grows above the gum line (supragingival plaque) play no role in periodontal disease. A relatively small number of bacterial species that reside within plaque on the tooth surface below the gum line (subgingival plaque) cause both gingivitis and the more severe periodontitis which typically results if gingivitis is left untreated. Periodontitis begins with enlargement of the space between the gums and the teeth forming a periodontal pocket which facilitates the further expansion of the subgingival plaque microflora in a low oxygen tension environment. If untreated, periodontitis may result in progressive resorption of alveolar bone, increasing mobility of the teeth and in the advanced stages tooth loss.

It is estimated that half the adult population of the United States has gingivitis and 30% have evidence of periodontitis on an average of 3 to 4 teeth (Oliver et al 1998). These are chronic conditions which may progress over several years with episodes of exacerbation and remission. Periodontitis can also occur with higher frequency in patients suffering from systemic diseases such as diabetes mellitus, AIDS, leukemia, neutropenia, Crohn's disease and Down's syndrome (Vrotos & Vrahopoulos, 1996; Kuo et al, 2007). Advanced adult periodontitis, leading to severe loss of supporting tissues, has a prevalence of 5-15% in most populations (Papapanou et al, 1999; Oliver et al, 1998; Albandar et al, 1999) and is a major cause of tooth loss in adults. Signs of gingivitis are common in young adults but the prevalence of periodontitis increases with age. Thus periodontal disease represents a considerable public health problem that can impact on general health status.

Gingivitis is characterised by one or more of the following symptoms: inflammation of the gingiva resulting in redness, swelling, shine and tenderness when pressure is applied and a propensity to bleed easily especially when the teeth are brushed or during flossing. When gingivitis progresses to the more severe periodontitis additional symptoms include formation of periodontal pockets; bleeding and/or pus discharge from the periodontal pockets; bite changes; pain on chewing; resorption of alveolar bone; loose teeth; and loss of teeth sometimes accompanied by a bad taste in the mouth or breath malodour.

The development of plaque is natural and inevitable. Plaque develops stepwise over time and depends on the ability of certain oral bacteria to form biofilms (Overman, 2000). A biofilm is produced when one or more species of bacteria adhering to a surface secrete polymeric substances in which they become embedded. The first stage in formation of the plaque biofilm is the coating of the tooth surface with proteins and glycoproteins, mainly from saliva to form a sticky film (pellicle). Early colonising bacteria can attach to this film directly. Late colonisers cannot but must attach to bacteria already present via specific molecular interactions. The resulting plaque is thus a complex of numerous bacterial species embedded in a matrix of polymers of bacterial and salivary origin. The matrix helps to sequester the bacteria from the inhibitory effects of antimicrobials that are unable adequately to penetrate the biofilm.

The specific plaque hypothesis states that there are two types of dental plaque. The first type, non-diseased plaque, is associated with good dental hygiene and good dental health. However, poor dental hygiene, and genetic, environmental and behavioral factors can encourage a shift in the bacterial composition to a cariogenic supragingival plaque or a periodontopathic subgingival plaque. Periodontopathic subgingival plaque although derived initially from supragingival plaque consists predominantly of bacteria that are the metabolic opposites of those involved in dental caries. These bacteria are strict anaerobes that thrive in the periodontal pocket where oxygen-dependent species cannot survive.

Periodontal disease is now considered to result from a polymicrobial infection associated with a relatively small number of highly pathogenic anaerobic bacteria. Of the numerous bacterial species that live in the oral cavity, less than twenty are strongly implicated in gingivitis and/or periodontitis. A further twenty species may have a minor or as yet unspecified role. Periodontal pathogens can be transmitted between individuals both vertically and horizontally.

Although the clinical signs and symptoms of periodontal diseases differ, Porphyromonas gingivalis, Tannerella forsythia and Treponema denticola are invariably found, occasionally with species belonging to such genera as Prevotella, Eubacterium, Fusobacterium and Peptostreptococcus. Aggretibacter actinomycetencomitans is an uncommon and opportunistic periodontal pathogen. Together, P. gingivalis, T forsythia and T. denticola form the ‘red complex’, the climax community within the subgingival plaque at sites showing evidence of progressive periodontitis (Holt & Ebersole, 2005).

Periodontal pathogens tend to be motile, slow growing Gram-negative species (except Eubacterium and Peptostreptococcus which are non-motile and Gram-positive). Unlike the pathogenic streptococci of supragingival plaque which utilise dietary sugars as their main carbon and energy source, periodontal pathogens rely more on proteins of both dietary (trapped food particles) and host origin.

Clinical evidence connecting P. gingivalis with periodontal disease is overwhelming and has been extensively reviewed (Lamont & Jenkinson 1998; Holt & Ebersole 2000). Elevated levels of the organism are found in periodontal lesions, and low levels in healthy sites. The bacterium is often eliminated by successful therapies but is found in recurrent infections. It produces an array of putative colonisation and virulence factors that facilitate adherence to, as well as invasion and destruction of, a number of periodontal cell and tissue types. A key feature of the organism is its ability to perturb the structural and functional integrity of the non-keratinised junctional epithelium (the crevice between the free gingival margin and the tooth) that plays a vital role in tissue homeostasis and innate antibacterial defence (Bosshardt & Lang, 2005).

Many of the end products of metabolism of P. gingivalis (such as ammonia, organic acids and volatile sulphur compounds) are cytotoxic to these epithelial cells. More specific agents of host tissue damage include the gingipains, powerful cysteine proteinases, which so far have not been detected in any other bacterial species. They mediate tissue damage both directly via their enzymatic functions and indirectly via modulation of the immune response. P. gingivalis subverts the host's immune response in a number of ways, for example by interfering with the functioning of the local cytokine network. As a result, a cascade of events initially triggered by bacterial factors and exacerbated by the dysregulated host response to them leads to self-destruction of the tissues supporting the teeth. Whilst P. gingivalis is a sophisticated pathogen, its virulence seems to be intensified in the presence of a microbial consortium that includes T. forsythia and T. denticola.

Thus whilst good oral hygiene, for instance regular brushing of the teeth with an appropriate cleansing product, may help to reduce the impact of periodontal disease, it does not necessarily eliminate its occurrence because micro-organisms contribute to both the initiation and progress of the disease. In order to prevent or treat periodontal diseases effectively, it is necessary to use a combination of approaches that compliment physical methods with the use of compounds that disrupt or prevent biofilm formation and compounds that kill pathogenic bacteria within plaque (Teles et al, 2006). Towards this end, there has been a great deal of research aimed at developing antimicrobially active dentifrices, mouthwashes and other similar products for combating periodontal disease. The antimicrobial agents included in such products must be able to access bacteria within plaque and retain their biological activity in the presence of a variety of interfering substances of dietary and host cell origin.

As with many chronic infections, prevention is extremely important to reduce both the prevalence and severity of periodontal disease. Antibiotics are used to treat established infection but have no role in prevention due to concerns about resistance in targeted and non-targeted bacteria arising from long term or repeated use. Often, metronidazole is preferred to other antibiotics because it is specifically active against anaerobic bacteria and leaves the healthy commensal flora intact. Tetracyclines such as doxycycline are sometimes used because, in addition to their antibacterial effects, they possess direct anti-inflammatory effects (eg anti-collagenase activity) that limit tissue damage.

Strategies aimed at preventing periodontal disease focus on improved dental hygiene (better brushing techniques, use of dental floss, regular visits to a dental hygienist) and the incorporation of antibacterial agents into toothpastes and mouthwashes. The two most frequently used antibacterial agents are chlorhexidine (a biguanide) and triclosan (a diphenyl ether derivative). Plant derived components such as thymol and sanguinarine, as well as quarternary ammonium compounds such as cetylpyridinium chloride are also used, especially in mouthwashes, whilst metal salts are commonly used in toothpastes. These agents are characterised by a broad spectrum of activity against oral bacteria, including pathogens and non-pathogens. Some of them, in particular chlorhexidine, are also used in varnishes, gels and implants for treating established disease, often as an adjunct to antibiotic therapy.

To be successful, agents used in oral healthcare must be delivered to and retained in plaque and must remain active long enough to target bacterial cells embedded in the extracellular matrix (Brading & Marsh, 2003).

Because of the continuing high prevalence of periodontal disease, despite the above described measures, there is an ongoing need for alternative and preferably improved treatments, both therapeutic and preventative, for the condition. Such treatments should be effective against the relevant micro-organisms, and/or in inhibiting plaque formation, and should ideally be suitable for local application so as to avoid the potentially undesirable side effects which can be associated with systemic treatments. They should also, preferably, be suitable for administration by the patient himself.

Certain benzo- and hydroquinones, in particular t-butyl hydroquinone (TBHQ), are known for use as antioxidants and in some contexts as antimicrobial agents. TBHQ itself for example has been used as a preservative to stabilise foodstuffs, cosmetics and even adhesives. It has also been recognised as an antimycotic (DE-44 34 312).

WO-2006/100496 describes the use of TBHQ, and other optionally substituted benzo- and hydroquinones, as antimicrobial agents for the topical treatment of skin and skin structure conditions such as acne. It demonstrates the in vitro activity of some of these quinones against propionibacteria such as P. acnes and against staphylococci such as S. aureus. It does not however demonstrate activity against the bacteria known to be associated with periodontal diseases, in particular against P. gingivalis.

Other more complex quinones of various types have been disclosed for use as antimicrobial agents—see JP-2003-267910, JP-09-255547, JP-04-211646, JP-04-211644, U.S. Pat. No. 6,228,891, DE-199 11 680 and GB-1,133,897—and as preservatives (JP-02202804 and GB-865,808). Ubiquinones, such as 2,3-dimethoxy-5-methyl-1,4-benzoquinone and “coenzyme Q10”, have been disclosed for use against P. gingivalis and for the treatment of halitosis and periodontal disease (see WO-03/037284, WO-2006/110183 and WO-00/33802).

Kupp et al in J. Dent. Res., 64(7): 1016-1018 present in vitro data for the activity of TBHQ against Streptococcus mutans (S. mutans), in particular its ability to inhibit both growth and lactic acid production by certain cariogenic strains of the organism. Himejima et al in Bioorganic & Medicinal Chemistry, 12 (2004): 921-925 also disclose the ability of hydroquinone, benzoquinone, resorcinol, catechol and arbutin to alter sucrose-induced adherence of S. mutans.

S. mutans is a Gram-positive facultative bacterium known to cause dental caries. However it differs in several key respects from P. gingivalis, including its structure, its behaviour and its mode of action: in particular its location is supra-gingival rather than sub-gingival, it is an adherent rather than motile species, its metabolic activity is saccharolytic rather than proteolytic and its growth rate (doubling time) is 4 to 5 hours as opposed to the days to weeks which P. gingivalis takes to grow to the same extent. Dental caries and periodontal disease are therefore considered to be two very distinct and in many respects contrasting conditions, as for instance explained by Loesche in Infect. Dis. Clin. N. Am., 21 (2007): 471-502, periodontal diseases being dominated by proteolytic and anaerobic bacteria.

Thus one would not necessarily expect compounds which have been shown to be active in vitro against S. mutans also to be active against P. gingivalis, or to be of use in tackling periodontal diseases.

Moreover the activity demonstrated by Kupp et al is not a reliable predictor of activity in vivo in the mouth, as the authors themselves admit at the end of their discussion, in particular since in this environment both S. mutans and P. gingivalis form biofilms which could significantly impact upon the ability of a potential antibacterial agent to inhibit or kill the bacteria.

It has now surprisingly been found that alkyl- and halo-substituted benzo- and hydroquinones such as TBHQ can be extremely active against P. gingivalis, even in the presence of biofilms, and can therefore be used to treat periodontal diseases such as gingivitis.

### STATEMENTS OF THE INVENTION

According to a first aspect of the present invention there is provided an alkyl- or halo-substituted benzoquinone, hydroquinone or mixture thereof, for use in the treatment of a periodontal disease, and/or for use in the treatment of a condition which is caused by, transmitted by and/or exacerbated by (typically either caused or transmitted by) P. gingivalis.

A second aspect provides the use of an alkyl- or halo-substituted benzoquinone, hydroquinone or mixture thereof in the manufacture of a medicament for the treatment of a periodontal disease, and/or for the treatment of a condition which is caused by, transmitted by and/or exacerbated by (typically either caused or transmitted by) P. gingivalis.

The present invention will typically involve the use of the benzoquinone, hydroquinone or mixture thereof as an antibacterial agent. It may involve use of the benzoquinone, hydroquinone or mixture thereof as an anti-plaque agent.

Periodontal diseases which may be treated using the present invention (the term “treat” embracing both therapeutic and prophylactic measures, as defined below) include for example dental plaque-induced gingival diseases; chronic (previously adult) periodontitis; aggressive periodontitis (formerly early-onset, prepubertal, juvenile or rapidly progressive periodontitis); necrotising periodontal diseases; abcesses of the periodontium; and post-operative bacterial infections (in particular those which are caused, transmitted and/or exacerbated by P. gingivalis). The invention may also be used to treat bacterial infections (in particular those which are caused, transmitted and/or exacerbated by P. gingivalis) of wounds or other lesions within the oral cavity, including those arising due to other medical conditions such as oral candidiasis.

Associated symptoms which may be treated using the invention include mouth ulcers, dental pain, discomfort, inflammation, bleeding, pus secretion, halitosis, tooth mobility, tooth loss, swelling or inflammation caused by any of the foregoing.

Since plaque formation on tooth surfaces can also cause, exacerbate or accompany periodontal diseases, in accordance with the present invention the benzoquinone, hydroquinone or mixture thereof may be used to reduce or prevent plaque formation, and/or to alter (suitably beneficially) the bacterial composition of plaque.

It has further been found that periodontal inflammatory diseases may be linked to (ie may in cases cause, increase susceptibility to and/or exacerbate) other more serious, often more systemic conditions. For example, periodontal diseases, and/or the by-products generated by associated pathogens such as P. gingivalis, have been linked with coronary artery disease and other cardiovascular diseases such as myocardial infarction, atherosclerosis and angina, as well as with conditions associated with arterial inflammation or blood clot formation and with an increased risk of pre-term low-weight births (Gotsman et al, J Periodontol. May 2007, 78(5): 849-858; Noack et al, J. Periodontol. 2001, 72: 1221-1227; Seymour et al, Clinical Microbiology and Infection, 13 (Suppl 4): 3-10). The present invention may accordingly be used, indirectly, to treat any such condition as well as to treat infections within the oral cavity and associated periodontal diseases.

It has also been recognised that periodontal diseases can pose a threat to the health of those suffering from chronic diseases such as diabetes, respiratory diseases, osteoporosis and AIDS (Kuo et al, Seymour et al, supra). Thus the present invention may be used to reduce health risks to such patients from actual or potential periodontal infections.

It has surprisingly been found that alkyl- and halo-substituted benzo/hydroquinones, such as TBHQ, can be highly active against P. gingivalis bacterial strains. This activity appears to be highly selective, ie to be significantly higher than the activity of the same compounds against other bacteria found in the oral cavity, for example S. mutans. It can also be very much higher than the activity of the same compounds against other Gram-negative bacteria.

Moreover the activity can in cases be superior to that observed for the antibacterial agents previously used to combat periodontal diseases, for example chlorhexidine. It appears that P. gingivalis is exceptionally sensitive to such quinone antimicrobial agents, an effect which could not have been predicted from the prior art and indeed the underlying mechanism for which may not yet be properly understood.

Certain alkyl- or halo-substituted benzo/hydroquinones have also been found to be effective against P. gingivalis when grown as a biofilm on hydroxyapatite, simulating the conditions of their intended use in vivo in the oral cavity.

Thus according to a third aspect of the present invention there is provided an alkyl- or halo-substituted benzoquinone, hydroquinone or mixture thereof, for use in disrupting and/or suppressing biofilm formation by P. gingivalis in the mouth.

Moreover in the context of the first and second aspects of the invention, a condition which is caused, transmitted and/or exacerbated by P. gingivalis may be caused, transmitted and/or exacerbated by biofilm formation by P. gingivalis.

According to a fourth aspect, the invention provides the use of an alkyl- or halo-substituted benzoquinone, hydroquinone or mixture thereof in the manufacture of a medicament for disrupting and/or suppressing biofilm formation by P. gingivalis in the mouth.

In the present context, the disruption of biofilm formation embraces any negative effect on the ability of the relevant micro-organism to form biofilms, and/or on a biofilm already formed by the organism. Thus, it may involve reducing the amount of a previously formed biofilm, and/or impairing such a biofilm, the biofilm typically being in the form of dental plaque. The disruption may involve altering the bacterial composition of dental plaque to a more healthy state, reducing the amount of plaque by for example inhibiting co-aggregation, and/or interfering with the production of signal molecules that mediate the organisation and integrity of plaque. It may involve killing or inhibiting sessile bacteria within a biofilm.

Suppression of biofilm formation embraces any degree of impairment (including complete prevention) of the ability of the micro-organism to form, or more typically to co-aggregate with, biofilms. It thus embraces total or partial impairment, including reducing the amount and/or strength of biofilm which the organism is able to form and/or the speed with which it is able to do so. It may involve preventing or reducing the growth or the rate of growth of an existing biofilm formed by the organism or by another organism such as Streptococcus gordonii, Streptococcus mitis, Actinomyces naeslundii or Fusobacterium nucleatum.

A fifth aspect of the invention provides an alkyl- or halo-substituted benzoquinone, hydroquinone or mixture thereof for use in inhibiting, at least partially, the ability of P. gingivalis to invade junctional epithelia in the mouth, and/or to attach to or be internalised by non-keratinised epidermal cells in the mouth.

In the context of the present invention, the benzoquinone, hydroquinone or mixture thereof will typically be used in the form of a pharmaceutical (which term includes veterinary) formulation, which is preferably suitable for topical application to tissue surfaces within the mouth, in particular the gums, gingivae, periodontium and/or teeth. The benzo/hydroquinone is therefore preferably contained in a pharmaceutically acceptable vehicle which can safely be applied to, and/or contacted with, such surfaces. A formulation which is “suitable for” topical application may also be adapted for topical application. Such a formulation may in particular be suitable for human use.

A formulation which is applied topically will not, in the course of its ordinary useage, be intentionally swallowed for the purpose of systemically administering a substance contained within it, but rather, will be retained in the oral cavity, in contact with the relevant oral tissue and/or dental surface, for sufficient time as to exert a pharmaceutical effect, in this case an antibacterial and/or anti-plaque effect.

Suitable vehicles for use in such formulations will be well known to those skilled in the art of preparing topical pharmaceutical preparations. The vehicle will typically be a fluid, which term includes a cream, paste, gel, lotion, foam, ointment, varnish or other viscous or semi-viscous fluid, as well as less viscous fluids such as might be used in sprays, drops, aerosols or mouthwashes.

The benzo/hydroquinone may be present in the form of a solution or suspension, the term “suspension” including emulsions, micellar systems and other multi-phase dispersions.

The benzo/hydroquinone may in general, however, be delivered by any appropriate route, whether local or systemic. It may for example be delivered orally, for instance in the form of a tablet, capsule, powder, granules, solution or suspension. In this case the quinone should be used in the form of a formulation which is suitable and/or adapted for oral ingestion. Again suitable vehicles for use in such formulations will be well known to those skilled in the art of preparing pharmaceutical preparations for oral delivery. Oral delivery may be particularly useful for the treatment of a systemic condition which is caused and/or exacerbated by P. gingivalis, for example coronary artery disease, cardiovascular diseases and conditions associated with arterial inflammation or blood clot formation. It may be used for example to administer a dose or series of doses of the benzo/hydroquinone, at levels suitable for preventing, or reducing the risk of, a subsequent P. gingivalis infection or a condition which could be caused or exacerbated by such an infection.

Alternatively the quinone may be delivered transdermally, for instance via a skin patch.

The benzo/hydroquinone may be carried in or on a delivery vehicle which is suitable for targeting or controlling its release at the intended site of administration. Such vehicles include liposomes and other encapsulating entities, for example niosomes, aspasomes, microsponges, microemulsions, hydrogels and solid lipid nanoparticles.

In the context of the present invention, the term “benzoquinone” means a cyclohexadiene dione (typically a cyclohexadiene-1,4-dione or cyclohexadiene-1,2-dione), or any similar compound containing two or more C═O groups in an unsaturated 6-membered hydrocarbon ring. The term “hydroquinone” (sometimes known as a “hydroxyquinone”) means a benzoquinone in which one or more (preferably both) of the C═O groups is instead present as a C—OH group, or as a radical group C—O^{•}. A hydroquinone may therefore be a compound having an unsaturated 6-membered hydrocarbon ring, typically a phenyl ring, which carries two or more —OH or —O^{•} groups.

A benzo/hydroquinone may be present as a mixture of two or more of these forms, for instance as an equilibrium mixture of a benzoquinone and its corresponding hydroquinone.

The two C═O groups or C—OH groups of a benzo- or hydroquinone may be positioned ortho, meta or para to one another. When positioned ortho to one another, this is known as a cyclohexadiene-1,2-dione or o-benzoquinone or, in the case of the corresponding hydroquinone, a catechol. When positioned meta to one another, this is known as a cyclohexadiene-1,3-dione or an m-benzoquinone or, in the case of the corresponding hydroquinone, a resorcinol. When positioned para to one another, this is known as a cyclohexadiene-1,4-dione or a p-benzoquinone or, in the case of the para-substituted HO-Ph-OH, simply as “hydroquinone”.

Preferably the two C═O groups or C—OH groups are positioned ortho or para to one another, most preferably para.

The term “hydroquinone” is not intended to embrace phenols, which have only one —OH group attached to a six-membered hydrocarbon ring.

In accordance with the present invention, the alkyl- or halo-substituted benzo/hydroquinone may be a mixture of an alkyl- or halo-substituted benzoquinone and its corresponding hydroquinone. It may be an alkyl- or halo-substituted hydroquinone. As mentioned above, a hydroquinone may be present in the form of a radical in which one or more of the C—OH groups exists as C—O^{•}.

Such compounds may be substituted with one or more alkyl groups, in particular C₁ to C₆ or C₁ to C₄ alkyl groups, for instance methyl, ethyl, isopropyl or t-butyl groups. Instead or in addition, they may be substituted with one or more halo groups. They are not generally substituted with other, non-alkyl and non-halo substituents, in particular alkoxyl groups such as methoxyl groups.

The quinone may include up to six or more suitably four alkyl groups, but in particular may be mono- or di-substituted with such groups, more preferably mono-substituted.

The quinone may be either an alkyl-substituted benzoquinone or an alkyl-substituted hydroquinone, or a mixture of an alkyl-substituted benzoquinone and an alkyl-substituted hydroquinone. More preferably it is an alkyl-substituted hydroquinone.

In the context of the present invention, an alkyl group may be either a straight or a branched chain alkyl group, of which the latter may be preferred, especially where the number of carbon atoms is 3 or greater. It may be or contain cycloalkyl moieties. It may contain for instance from 1 to 12 carbon atoms, preferably from 1 to 10, more preferably from 1 to 8. Particularly preferred alkyl groups are those selected from C₁ to C₆ alkyl groups, more preferably C₁ to C₅ alkyl groups, yet more preferably C₁ to C₄ alkyl groups, for instance methyl, ethyl, iso-propyl or t-butyl groups.

In an alkyl-substituted hydroquinone, an alkyl substituent may be attached to a carbon atom of the cyclohexyl ring or to an oxygen atom (thus replacing the hydrogen atom of a hydroxyl group on the cyclohexyl ring). Preferably it is attached to a carbon atom.

A hydroquinone may thus be substituted with up to six alkyl groups, more preferably up to four alkyl groups, but in particular may be a mono- or di-alkyl hydroquinone, preferably the former.

The hydroquinone may be substituted with one butyl group, which is preferably present at the 2 position; it may however be substituted with more than one butyl group, for instance two or three or four. A butyl group is preferably a t-butyl group.

Instead or in addition, the hydroquinone may be substituted with one hexyl group, which is preferably an O-substituted hexyl group replacing the hydrogen atom of a hydroxyl group. The hydroquinone may however be substituted with more than one hexyl group, for instance two or three or even four. A hexyl group may be a straight chain hexyl group.

Instead or in addition, the hydroquinone may be substituted with one methyl group, which is preferably present at the 2 position; it may however be substituted with more than one methyl group, for instance two or three or four or even five. It may for instance be substituted with three methyl groups, which are preferably present at the 2, 3 and 5 positions.

Instead or in addition, the hydroquinone may be substituted with one propyl group, which is preferably present at the 2 position. The hydroquinone may however be substituted with more than one propyl group, for instance two or three or four. A propyl group is suitably an iso-propyl group.

Instead or in addition, the hydroquinone may be substituted with one ethyl group, which is preferably present at the 2 position. The hydroquinone may however be substituted with more than one ethyl group, for instance two, three, four or even five.

Instead or in addition, the hydroquinone may be substituted with one, two, three or even four pentyl (preferably t-amyl) groups. In an embodiment it is substituted with two t-pentyl groups, which preferably occupy the 2 and 5 positions.

In an embodiment the hydroquinone may be substituted with three methyl groups and one hexyl group, the hexyl group preferably replacing the hydrogen atom of a hydroxyl group and the three methyl groups preferably occupying the 2, 3 and 5 positions.

The hydroquinone may be substituted with one methyl and one iso-propyl group, which preferably occupy the 5 and the 2 positions respectively.

In particular the hydroquinone may be substituted with just one t-butyl group, which is preferably present at the 2 position. Alternatively the hydroquinone may be substituted with two butyl groups, which preferably occupy the 2 and 5 positions. In both cases the butyl groups are preferably t-butyl groups.

In certain cases it may be preferred for the hydroquinone not to be a resorcinol. In some cases it may be preferred for the hydroquinone not to be a catechol.

In some cases it may be preferred for the hydroquinone not to be a C₆ to C₉ alkyl-substituted resorcinol, in particular n-hexylresorcinol.

A halo-substituted hydroquinone may be substituted with up to four halo groups, more preferably up to three halo groups, but in particular may be a mono- or di-halo hydroquinone. A halo group may be for example either fluoro, chloro, bromo or iodo, suitably either chloro or fluoro, more suitably chloro. A hydroquinone may be substituted with one or more halo groups and in addition with one or more alkyl groups of the type described above.

An alkyl- or halo-substituted hydroquinone may be selected from the group consisting of 2-t-butyl-p-hydroquinone (TBHQ), 2,5-di-t-butyl-p-hydroquinone, 2,5-di-t-pentyl-p-hydroquinone, 2-isopropyl-p-hydroquinone, 2-ethyl-p-hydroquinone, 2-methyl-p-hydroquinone, 4-hexyl resorcinol and mixtures thereof. In cases it may be thymohydroquinone, which is a para-hydroquinone substituted at the 2-position with an isopropyl group and at the 5-position with a methyl group. In cases it may be 2,3-difluoro-p-hydroquinone. It may be selected from the group consisting of TBHQ, 2,5-di-t-butyl-p-hydroquinone, 2-ethyl-p-hydroquinone, 2-methyl-p-hydroquinone, 2,5-di-t-pentyl-p-hydroquinone, thymohydroquinone, 4-hexyl resorcinol, 2,3-difluoro-p-hydroquinone and mixtures thereof. It may in particular be TBHQ, ie a para-hydroquinone substituted at the 2 position with a t-butyl group.

An alkyl-substituted benzoquinone may be substituted with one or more alkyl groups, an alkyl group being as defined above. Substituents on a benzoquinone will be attached to carbon atoms of the cyclohexyl ring.

A benzoquinone may be substituted with up to four alkyl groups, but in particular may be a mono- or di-alkyl benzoquinone, preferably the former.

Such a benzoquinone is preferably substituted with one methyl group, which is preferably present at either the 2 or the 5 position; it may be substituted with more than one methyl group, for instance two or three or even four.

Instead or in addition, the benzoquinone is preferably substituted with one propyl group, which is preferably present at the 2 position; it may be substituted with more than one propyl group, for instance two or three or even four. A propyl group is preferably an iso-propyl group.

In particular the benzoquinone may be substituted with one methyl and one iso-propyl group, which preferably occupy the 5 and 2 positions respectively.

The benzoquinone may be substituted with one butyl group (for instance at the 2 position), or with more than one (for instance two, three or four) butyl groups. A butyl group is preferably a t-butyl group.

The benzoquinone may be substituted with two butyl groups, either or preferably both of which is a t-butyl group. These may for instance occupy the 2 and 5 positions, in particular where the benzoquinone is a para-benzoquinone. They may alternatively occupy the 3 and 5 positions, in particular where the benzoquinone is an ortho-benzoquinone.

Instead or in addition, the benzoquinone is preferably substituted with one ethyl group, which is preferably present at the 2 position; it may be substituted with more than one ethyl group, for instance two or three or even four.

Instead or in addition, the benzoquinone may be substituted with one, two, three or even four pentyl (preferably t-amyl) groups.

Instead or in addition, the benzoquinone may be substituted with one, two, three or even four hexyl groups.

A halo-substituted benzoquinone may be substituted with up to four halo groups, more preferably up to three halo groups, but in particular may be a mono- or di-halo hydroquinone. A halo group may be for example either fluoro, chloro, bromo or iodo, suitably either chloro or fluoro, more suitably chloro. A benzoquinone may be substituted with one or more halo groups and in addition with one or more alkyl groups of the type described above.

An alkyl- or halo-substituted benzoquinone may be selected from the group consisting of 2-t-butyl-p-benzoquinone (also known simply as t-butyl benzoquinone, or TBBQ), 2,5-di-t-butyl-p-benzoquinone, 2-ethyl-p-benzoquinone, 2-methyl-p-benzoquinone and mixtures thereof. In cases it may be thymoquinone, which is a para-benzoquinone substituted at the 2-position with an iso-propyl group and at the 5-position with a methyl group. It may be 2-chloro-5-methyl-p-benzoquinone. It may be selected from the group consisting of TBBQ, 2-ethyl-p-benzoquinone, 2-methyl-p-benzoquinone, thymoquinone, 2-chloro-5-methyl-p-benzoquinone and mixtures thereof. It may in particular be TBBQ.

In an embodiment of the invention, the quinone is an alkyl-substituted benzoquinone, hydroquinone or mixture thereof. In another embodiment, it is selected from the group consisting of TBHQ, TBBQ and mixtures thereof.

In an embodiment of the invention it may be preferred for the quinone not to be thymoquinone or thymohydroquinone.

In the present context an alkyl- or halo-substituted benzo/hydroquinone may be present in the form of a dimer, oligomer or polymer, the monomer unit of which is an alkyl- or halo-substituted benzo/hydroquinone as defined above. It may be in the form of a pharmaceutically acceptable (which term includes acceptable for veterinary use) derivative, for example a salt, complex or solvate or a so-called “prodrug” form or protected form which reverts to an active form of the relevant compound at an appropriate time on or after administration. Preferably, however, the benzo/hydroquinone is present in the form of a single, underivatised benzo/hydroquinone molecule.

A benzo/hydroquinone used in a formulation prepared according to the invention, in particular thymoquinone, dithymoquinone or thymohydroquinone, is ideally used in the form of the isolated quinone (whether naturally or synthetically derived, preferably the latter) rather than as part of a plant extract containing a number of different materials.

The benzo/hydroquinone may be of the type which is active as an antioxidant.

Most preferred quinones for use according to the invention are those selected from TBHQ, TBBQ and mixtures thereof. In particular the quinone may be TBHQ.

According to the invention, a mixture of two or more alkyl- or halo-substituted benzo/hydroquinones as described above may be used against P. gingivalis or in the treatment of a condition which is caused by, transmitted by and/or exacerbated by P. gingivalis.

According to the invention, the benzo/hydroquinone is preferably used as an antibacterially active agent against P. gingivalis. It may be used as an active agent against biofilm formation by P. gingivalis.

Antibacterial activity may be growth inhibitory activity or more preferably biocidal (ie lethal to the relevant organism). It may comprise activity against sessile and/or planktonic bacteria.

In the context of this invention, activity against a particular species of bacterium may be taken to mean activity against at least one, preferably two or more, strains of that species.

Antibacterial activity may be measured in conventional manner, for instance using the tests described in the examples below. Generally tests for activity involve treating a culture of the relevant micro-organism with the candidate antibacterial compound, incubating the treated culture under conditions which would ordinarily support growth of the organism, and assessing the level of growth, if any, which can occur in the presence of the candidate compound.

Activity against P. gingivalis may in particular be assessed in the presence of an established biofilm formed by the bacterium. Activity in suppressing or disrupting biofilm formation by P. gingivalis may be assessed for instance as described in the examples below, suitably on a hydroxyapatite substrate.

Preferably the alkyl- or halo-substituted benzo/hydroquinone used in the present invention has a minimum inhibitory concentration (MIC), against P. gingivalis, of 5 μg/ml or less, preferably 2 μg/ml or less, such as from 0.25 or less to 1 μg/ml. Suitably the ratio of its MIC to its corresponding minimum biocidal concentration (MBC) is from 0.125 to 1, ideally from 0.5 to 1. More preferably the benzo/hydroquinone also exhibits such characteristics in the presence of at least one of, preferably both of, saliva and gingival crevicular fluid, and/or in the presence of a component of such a fluid such as mucin or albumin—these are species which can be present in the mouth and hence performance in this context can be indicative of suitability for use in topical oral health care formulations.

MIC and MBC values may be measured using conventional assay techniques, for instance as described in the examples below.

The concentration of the benzo/hydroquinone in a formulation prepared according to the invention, in particular a formulation for topical and/or local delivery, might suitably be 0.01 or 0.05% w/v or greater, preferably 0.1% w/v or greater. Its concentration might be up to 10% w/v, preferably up to 5 or 2 or 1% w/v, such as from 0.1 to 1% w/v.

For oral delivery, the benzo/hydroquinone may be formulated in dosage forms—for example tablets or capsules—containing 2 mg or greater, preferably 5 or 10 or 20 mg or greater, of the active substance. Such dosage forms may contain up to 250 mg, or in cases up to 100 mg, of the active substance, for instance from about 2 to 250 mg or from about 10 to 100 mg.

As described above, a medicament (typically a formulation) prepared according to the invention is preferably suitable for, and more preferably adapted for, topical administration to the teeth, gums, skin or other tissue surfaces within the human or animal, in particular human, mouth. It may take the form of a lotion, cream, ointment, varnish, foam, paste or gel, or any other physical form known for topical administration. It may take the form of a solution or suspension, for instance for use as a mouthwash or as a disinfectant. It may take the form of an aerosol formulation or spray. It may comprise a formulation which is, or may be, applied to a carrier such as a sponge, swab, brush, tissue, cloth, wipe, skin patch, pad, dressing or dental fibre to facilitate its topical administration. It may comprise a formulation which is, or may be, carried on or in an implant (including for example a chip for insertion into a periodontal pocket; or a dental filling, bridge or cap; or a denture), an impregnated dental fibre or a chewing gum, tablet or chewable capsule.

The medicament may in particular take the form of a toothpaste, mouthwash, dentifrice, lozenge or buccal patch or a formulation carried in or on a dental fibre or tape. It may be intended for pharmaceutical (which includes veterinary but is preferably human) use, for example to treat bacterial infections within the oral cavity, or as a prophylactic against such infections, and/or for cosmetic or other non-medical care purposes (for example, for general hygiene or for improving the appearance of the teeth or gums).

In the case where such a formulation is intended for application to a non-living area or surface, for instance as a disinfectant, it may take the form of a solution or suspension of the benzo/hydroquinone in an appropriate fluid vehicle such as an alcohol or a water/alcohol mix. Again conventional excipients and other additives may be included, as may one or more additional antimicrobial (in particular antibacterial) agents.

In particular when the formulation is for use in controlling the transmission of a bacterial infection, it may be in the form of a skin wash (for example a hand wash), or of a surface disinfectant such as a spray, or of a cleansing fluid for example for use in disinfecting dentures or surgical (including dental) instruments. It may be carried in or on a cloth, wipe, brush or other cleaning utensil, or a substrate such as a preparation surface or implement or a packaging material; in such cases an item may be impregnated with, or coated with, the formulation.

The vehicle in which the benzo/hydroquinone is contained may be any vehicle or mixture of vehicles which is suitable for topical application; the type chosen will depend on the intended mode and site of application. Many such vehicles are known to those skilled in the art and are readily available commercially. Examples may for instance be found in “Oral Hygiene Products and Practice”, 1988, Morton Prader, Ed., Marcel Dekker, Inc., New York, N.Y., USA.

The benzo/hydroquinone may be present in the form of a suspension or other type of multi-phase dispersion, as described above.

Also as described above, the vehicle may be such as to target a desired site and/or time of delivery of the formulation. It may for instance target the formulation to the gums or teeth or other areas within the oral cavity. It may delay or otherwise control release of the formulation over a particular time period. The benzo/hydroquinone may be microencapsulated, for instance in liposomes.

In some cases a polar vehicle may be preferred. The vehicle may be primarily aqueous. The vehicle may be surface-active, in particular when it is intended for use in treating surfaces, for instance to cleanse instruments or working areas. It is suitably volatile, although in cases may be non-volatile.

A formulation prepared according to the invention may contain standard excipients and/or other additives known for use in pharmaceutical or veterinary formulations, in particular oral health care formulations. Examples include flavourings, antioxidants, preservatives, stabilisers, gelling agents and surfactants; others may be found in “Oral Hygiene Products and Practice”, 1988, supra. Surfactants may be particularly preferred, as they can help to disrupt, and/or prevent formation of, microbial biofilms.

The formulation should contain an orally acceptable and systemically non-toxic vehicle. For example, where it takes the form of a toothpaste, a typical vehicle might include water and a humectant to provide a liquid base, together with one or more of a thickener, a surfactant and a polishing agent. Suitable humectants include glycerol, sorbitol and polyethylene glycol, and in particular mixtures thereof. A polyethylene glycol humectant may for example have a molecular weight range of from 200 to 1000 or from 400 to 800, such as about 600.

Suitable thickeners for use in toothpaste formulations include natural and synthetic gums and colloids such as carrageenan, xanthan gum and sodium carboxymethyl cellulose, as well as gum tragacanth; starch; polyvinyl pyrrolidone; cellulosic thickeners such as hydroxyethyl propyl cellulose, hydroxybutyl methyl cellulose, hydroxypropyl methyl cellulose, hydroxyethyl cellulose and water soluble salts of cellulose ethers such as sodium carboxymethyl cellulose or sodium carboxymethyl hydroxyethyl cellulose; and carboxyvinyl polymers. Suitable inorganic thickeners include colloidal silica, colloidal magnesium aluminium silicate, finely divided silica and synthetic hectorite. Mixtures of thickeners may also be used.

Suitable surfactants for use in toothpaste formulations prepared according to the invention include water soluble detergents. In general they may be anionic, nonionic, cationic, zwitterionic, amphoteric or ampholytic, but are preferably anionic. Examples of suitable anionic surfactants include higher alkyl sulphates such as sodium lauryl sulphate, and higher fatty acid esters of 1,2 dihydroxy propane sulphonate. Examples of suitable water soluble nonionic surfactants include the polymeric condensation products of hydrophilic alkylene oxide group-containing compounds (typically ethylene oxide) with organic hydrophobic compounds (for example those having aliphatic chains of about 12 to 20 carbon atoms). Such products include the “ethoxamers” and include for example the condensation products of poly(ethylene oxide) with fatty acids, fatty alcohols, fatty amides and other fatty moieties, as well as with propylene oxide and polypropylene oxides (the latter being available, for example, under the trade name Pluronic®).

A toothpaste will suitably contain an abrasive or polishing agent. Suitable such agents include siliceous materials (including gels and precipitates, such as precipitated amorphous hydrated silicas, aluminium silicate, zirconlure silicate, silica gel and colloidal silica); carbonates and bicarbonates such as calcium carbonate and sodium bicarbonate; phosphates such as sodium metaphosphate, potassium metaphosphate, tricalcium phosphate, dicalcium orthophosphate dehydrate, calcium phosphate dihydrate, anhydrous dicalcium phosphate, calcium pyrophosphate, calcium polymetaphosphate, magnesium orthophosphate, trimagnesium phosphate and insoluble sodium polymetaphosphate; alumina trihydrate; calcined alumina; bentonite; complex amorphous alkali metal aluminosilicates; and resinous abrasive materials such as particulate condensation products of urea and formaldehyde. Others are disclosed in U.S. Pat. No. 3,070,510. Mixtures of such polishing agents may also be used. The abrasive or polishing agent should not excessively abrade tooth enamel or dentin.

Silica abrasive agents may be particularly preferred for use in the present invention.

Where a formulation prepared according to the invention takes the form of a mouthwash or dentifrice, it may for example contain a water/alcohol (eg water/ethyl alcohol) solution and optionally one or more other ingredients selected for example from flavourings, sweeteners, humectants, surfactants, emulsifiers if necessary and mixtures thereof. Suitable humectants include those described above, in particular glycerol and sorbitol. One or more additional antibacterial agents may also be included.

Non-soap surfactants (for example nonionic, cationic or amphoteric surfactants) may be preferred for use in mouthwash formulations. Suitable nonionic surfactants include the condensation products of hydrophilic alkylene oxide group-containing compounds with organic hydrophobic compounds, as described above. Other suitable nonionic synthetic detergents include: the polyethylene oxide condensates of alkyl phenols; those derived from the condensation of ethylene oxide with the product resulting from the reaction of propylene oxide and ethylene diamine; the condensation products of aliphatic alcohols having from 8 to 18 carbon atoms with ethylene oxide; and the polyoxyethylene derivatives of fatty acid partial esters of sorbitol anhydride (for example the commercially available Tween® products).

Suitable cationic detergents include quaternary ammonium compounds, in particular those having one long alkyl chain of about 8 to 18 carbon atoms, for example lauryl trimethylammonium chloride, cetyl pyridinium chloride, cetyl trimethylammonium bromide, di-isobutylphenoxyethyldimethylbenzylammonium chloride, coconutalkyltrimethylammonium nitrite, cetyl pyridinium fluoride and the like.

Suitable amphoteric detergents include derivatives of aliphatic secondary and tertiary amines in which one of the aliphatic substituents contains from about 8 to 18 carbon atoms and one contains an anionic water solubilising group such as carboxylate, sulphate, sulphonate, phosphate or phosphonate.

Other suitable surfactants, for use in formulations according to the invention, may be found in McCutcheon's Detergents and Emulsifiers and in U.S. Pat. No. 4,051,234.

A formulation prepared according to the invention may contain one or more additional agents, for example selected from abrasives, bleaching agents, tooth whitening agents (eg peroxides or sodium perborate), surface active agents/detergents as described above, foaming agents, sources of fluoride ions or fluorine-containing ions, zinc salts, non-cariogenic sweeteners such as saccharin or aspartame or dextrose or levulose, other flavourings such as peppermint or spearmint or aniseed, menthol, desensitising agents, anti-tartar/sequestering agents or anti-calculus agents (for example metal salts such as zinc chloride, zinc acetate or zinc oxide; pyrophosphate salts such as alkali metal or ammonium pyrophosphates; or diphosphonates), sodium bicarbonate, anionic polycarboxylates, enzymes such as lactoperoxidases, humectants as described above, binders such as carboxyvinyl polymers, pH regulating buffers, preservatives, colours/dyes (for example chlorophyll or titanium dioxide), plant extracts, anti-plaque agents, additional antimicrobial (for example antifungal or antibacterial, especially antibacterial) agents and mixtures thereof.

Suitable sources of fluoride or fluorine-containing ions are water soluble fluorides such as water soluble alkali metal or alkaline earth metal fluorides, for example sodium, potassium and barium fluorides (in particular alkali metal fluorides); copper fluorides, such as cuprous fluoride; tin fluorides; fluorosilicates such as sodium or ammonium to fluorosilicate; fluorozirconates such as sodium or ammonium fluorozirconate; monofluorophosphates such as sodium or potassium monofluorophosphate; mono-, di- and tri-aluminium fluorophosphates; and fluorinated pyrophosphates such as fluorinated sodium calcium pyrophosphate.

It may be necessary to include a solvent or solubilising agent in a formulation prepared according to the invention, in order to solubilise another agent (in particular an active agent) present in the formulation. Suitable solvents/solubilising agents include flavour oils, polyethylene glycols (preferably those having molecular weights of from about 200 to about 600), propylene glycol, dipropylene glycol, methyl cellosolve, ethyl cellosolve, olive oil, castor oil, amyl acetate, ethyl acetate, glyceryl tristearate, benzyl benzoate and mixtures thereof.

Generally speaking a formulation prepared according to the invention may contain one or more agents which enhance the activity of another active agent present in the formulation, or reduce a side effect of such an active, or improve patient compliance on administration of the formulation. It may contain one or more agents which facilitate penetration of an active agent, in particular the benzo/hydroquinone, into microbial biofilms. It may contain one or more agents which control the site and/or rate of release of an active agent, in particular the benzo/hydroquinone, following administration.

In general an additional antimicrobial agent may be selected from the group consisting of biocides, disinfectants, antiseptics, antibiotics, bacteriophages, enzymes, anti-adhesins, immunoglobulins and mixtures thereof; it is preferably active as a bactericide, in particular against P. gingivalis and/or against one or more other bacteria associated with oral health problems.

It may however be preferred for the benzo/hydroquinone to be the only active agent in the formulation, or at least to be the only antibacterially active agent and/or the only agent active against P. gingivalis or against biofilm formation by P. gingivalis.

A formulation prepared according to the invention may be suitable for, more preferably adapted for, use in an area or on a surface other than living tissue, for instance to treat work surfaces or instruments or to disinfect dentures or other dental equipment. It may be suitable for application to non-living tissue (for instance for use as a preservative). In these cases the excipients, vehicles and/or other additives included with the benzo/hydroquinone may be different to those included in a topical oral health care formulation, but again may be conventional as known for use in such contexts. In particular the formulation may take the form of a concentrate which can be diluted to a suitable concentration (for example as described above) prior to use.

A formulation prepared according to the invention may be incorporated into, and hence applied in the form of, another product such as a toothpaste, mouthwash, dentifrice, dental gel or dental floss. It may in particular be incorporated into a toothpaste or mouthwash. It may be incorporated into a cleansing preparation, for example a hand wash or soap for use by dental surgeons prior to treating patients. The invention provides such a product.

The invention thus embraces the use of an alkyl- or halo-substituted benzo/hydroquinone in the manufacture of such a product for disrupting and/or suppressing biofilm formation by P. gingivalis, or for the treatment of a condition (in particular a condition in the mouth, more particularly a periodontal disease) which is caused by, transmitted by and/or exacerbated by (in particular either caused by or transmitted by) P. gingivalis.

In the context of the present invention, treatment of a condition encompasses both therapeutic and prophylactic treatment, of either an infectious or a non-infectious condition, in either a human or animal but in particular a human. It may involve complete or partial eradication of the condition, removal or amelioration of associated symptoms, arresting or slowing subsequent development of the condition, and/or prevention of, or reduction of risk of, subsequent occurrence of the condition. It will typically involve use of the alkyl- or halo-substituted benzo/hydroquinone as a bactericide and/or bacteriostatic agent.

The benzo/hydroquinone may be used for the therapeutic or prophylactic treatment of a condition within the oral cavity which is caused by, transmitted by and/or exacerbated by P. gingivalis, including by biofilm formation involving P. gingivalis. Examples include periodontal diseases such as those listed above, in particular gingivitis and periodontitis. The benzo/hydroquinone may be used for the creation and/or maintenance of fresh-smelling breath, ie for the treatment (which includes prevention) of halitosis. It may be used to modify the bacterial composition of dental plaque. It may be used to treat a bacterial infection which has occurred or has the potential to occur following a dental procedure such as a tooth extraction or other surgical procedure.

According to a sixth aspect, the present invention provides the use of an alkyl- or halo-substituted benzo/hydroquinone as defined above, as an antibacterial agent against P. gingivalis, in particular to suppress and/or disrupt biofilm formation by P. gingivalis. The use preferably involves the topical application of the benzo/hydroquinone, to either a living or a non-living surface but in particular to tissue within the mouth.

A seventh aspect of the invention provides a method for controlling the growth of P. gingivalis or of a biofilm produced by P. gingivalis, the method comprising applying, to an area or surface which is infected or suspected to be infected or capable of becoming infected with the organism or biofilm, an alkyl- or halo-substituted benzoquinone, hydroquinone or mixture thereof.

In this context, “controlling the growth” of a micro-organism or biofilm embraces inhibiting or preventing its growth, whether completely or partially, as well as killing or inactivating either completely or partially a culture of the organism. It also embraces reducing the risk of subsequent growth of the organism or biofilm in or on the area or surface being treated. The method of the invention may thus be used to treat an existing occurrence of the organism or biofilm or to prevent, or reduce the risk of, a potential subsequent occurrence.

Again the area or surface to which the benzo/hydroquinone is applied will typically be a living surface such as human tissue, in particular in the mouth. In this case the benzo/hydroquinone may be applied for therapeutic purposes or for non-therapeutic (eg purely cosmetic) purposes. Thus an eighth aspect of the invention provides a method of treatment of a human or animal patient suffering from or at risk of suffering from a condition which is caused by, transmitted by and/or exacerbated by (in particular either caused by or transmitted by) P. gingivalis, in particular by biofilm formation by P. gingivalis, the method involving administering to the patient a therapeutically or prophylactically effective amount of an alkyl- or halo-substituted benzoquinone, hydroquinone or mixture thereof.

In an embodiment of this ninth aspect of the invention, the condition is a periodontal disease such as gingivitis or periodontitis. The condition may be or involve the formation of dental plaque. In an embodiment, the benzo/hydroquinone is administered topically. It may be applied to tissue outside of the oral cavity, in particular to the skin, for instance to disinfect hands or to cleanse the area around the mouth prior to oral surgery.

Alternatively the benzo/hydroquinone may be applied to a non-living surface such as in a hospital or dental surgery. For example the method of the seventh aspect of the invention may be used to treat work surfaces, surgical or other instruments (in particular dental instruments, toothbrushes or other personal oral health care implements), surgical implants or prostheses (including dental components such as dentures, caps, bridges and filling materials), protective clothing such as surgical gloves, floors and walls and many other surfaces. The method may in particular be used to treat dental instruments and components, toothbrushes and other personal oral health care implements, work surfaces or protective clothing.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

The present invention will now be further described with reference to the following non-limiting examples and the accompanying figures, of which:

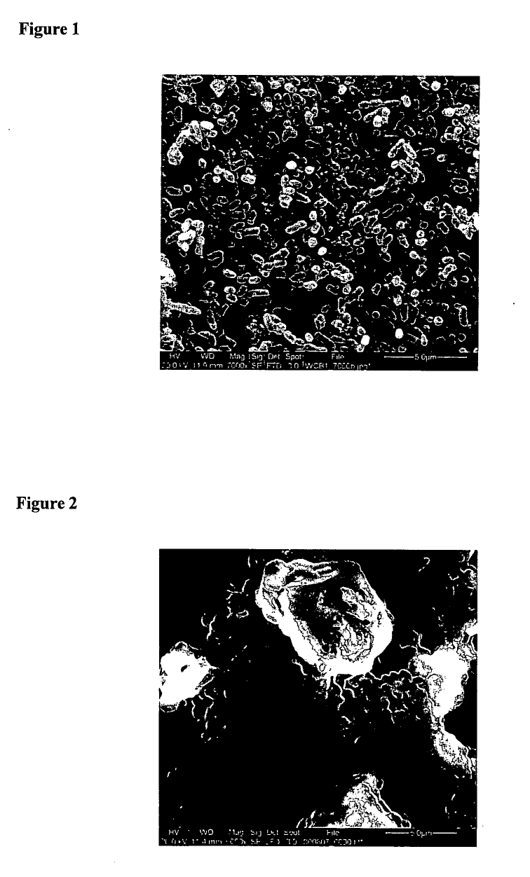

FIGS. 1 and 2 are scanning electron microscopy (SEM) images respectively of P. gingivalis 381 and S. mutans ATCC 25175 biofilms formed during the biofilm disruption assays of Example 3 below.

### DETAILED DESCRIPTION

Experimental tests were conducted to determine the antimicrobial activities of alkyl- and halo-substituted hydroquinones and benzoquinones against P. gingivalis.

Test Micro-Organisms

The principal test micro-organisms used were Porphyromonas gingivalis NCTC 11834 and P. gingivalis 381 (obtained from Professor H Kuramitsu, State University of Buffalo, Buffalo, N.Y., USA). These are black pigmented Gram-negative anaerobic bacteria belonging to the genus Porphyromonas. P. gingivalis is an oral pathogen typically associated with periodontal lesions, infections and adult periodontal disease. Gingivitis (inflammation of the gums that causes bleeding and exposes the base of the teeth) can be a precursor to periodontal disease by allowing P. gingivalis to infect the areas near the roots of the teeth and thus to cause tooth decay and infection.

Activity observed against these micro-organisms is expected to be a reasonable qualitative predictor of antimicrobial activity against micro-organisms responsible for periodontal lesions and infections and periodontal disease.

P. gingivalis strains were cultured and maintained on Wilkins-Chalgren Anaerobe Medium (agar and broth) at pH 7.0; all cultures were incubated anaerobically at 37° C. for 5-7 days.

Also tested was Streptococcus mutans ATCC 25175—this is a Gram-positive, microaerophilic bacterium associated principally with the human oral cavity. Clinically, S. mutans plays a significant role in dental caries and in infective endocarditis. The bacterium produces lactic acid as a by-product of its normal metabolism, and also produces an enzyme (dextransucrase) that can utilise sucrose to produce an extracellular dextran-based polysaccharide. This polysaccharide enables the bacteria to adhere to each other on the surface of a tooth to form plaque. It is the combination of the plaque and lactic acid that can result in tooth decay. More seriously, if the bacterium enters the bloodstream, for example after a tooth extraction, it can bind to the endocardium within the heart and if left untreated can prove fatal.

Activity observed against this micro-organism is expected to be a reasonable qualitative predictor of antimicrobial activity against micro-organisms responsible for dental caries and infective endocarditis.

S. mutans was cultured and maintained on Wilkins-Chalgren Anaerobe Medium (agar and broth) at pH 7.0 supplemented with 1 g/L glucose; all cultures were incubated at 37° C. for 48 hours in an atmosphere containing 5% CO₂. For the formation of biofilms in biofilm disruption assays, however, S. mutans was grown in brain-heart infusion broth supplemented with 5% sucrose.

The following tests were carried out to assess antimicrobial activity against the test organisms.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μl of liquid per well. The wells contained liquid culture medium and ranges of decreasing concentrations of the relevant test compound in doubling dilutions (eg 1000, 500, 250, 125 . . . μg/ml, etc down to 0.49 μg/ml). The culture medium was as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, ie the lowest concentration for which the liquid in the well remained clear.

The assays were conducted in duplicate (minimum) and included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Bactericidal Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target microbial population to recover.

(c) Short-Contact Kill Assay (SCKA)

This quantitative assay was designed to determine the relative potency of a test compound or combination of test compounds to kill a test micro-organism over a short-contact time period (up to 5 minutes).

Samples from a culture of micro-organisms (ca. 1×10⁸ cfu/ml) prepared in phosphate buffered saline (PBS) containing the relevant test compound(s) were taken at timed intervals (between 0 and 5 minutes), 10-fold serially diluted in PBS and then inoculated onto agar plates in triplicate. A culture containing only the solvent(s) used to dissolve the test compound(s) acted as a control for the assay (maximum solvent load 2.5% v/v).

The plates were then incubated as described above and subsequently examined visually for growth. Colonies were counted at an appropriate serial dilution (5-50 individual colonies visible) with the aid of a colony counter. These measurements were then converted to numbers of colony forming units (cfu), using the formula: cfu/ml=number of colonies×serial dilution factor×100 (as only a 10 sample was taken).

These cfu values were then converted into logᵢₙ values and plotted graphically against time of sample removal.

At each time point, samples were assessed in triplicate; the final cfu/ml value was an average (mean) of the three readings.

(d) Biofilm Disruption Assay

This assay was designed to determine quantitatively the relative potency of a test compound to disrupt (effectively kill) biofilms formed by a test organism on hydroxyapatite (HA) discs.

Biofilms were formed on sterile dense ceramic HA discs 7 mm diameter×1.8 mm thick (Clarkson Chromatography) by placing the discs in the appropriate liquid media (as described above) inoculated with the test organism (0.5×10⁸ cfu/ml). The HA discs were then incubated at 37° C. for ˜48 hours in a 5% CO₂ atmosphere (for S. mutans ATCC 25175) or ˜96 hours under anaerobic conditions (for P. gingivalis 381). Following the incubation period the supernatant was removed from the wells containing the HA discs and the discs were washed x3 with fresh media to ensure all planktonic cells were removed. The HA discs were then transferred to sterile bijoux bottles containing 1 ml of phosphate buffered saline (PBS) plus test compound(s) at the required concentration. After a set time period (usually 2 and/or 5 min), the HA disc was transferred to another sterile bijoux containing 1 ml of fresh growth media. Adherent cells were then harvested by vigorous vortexing for 2 minutes. A sample was then taken, serially diluted 10-fold in fresh growth media and inoculated onto agar plates in triplicate. Untreated HA discs, and HA discs added to PBS containing only the solvents used to dissolve the test compound(s), acted as controls.

The plates were then incubated as described above and subsequently examined visually for growth. Colonies were counted at an appropriate serial dilution (5-50 individual colonies visible) with the aid of a colony counter. These measurements were then converted to numbers of colony forming units (cfu), using the formula: cfu/ml=number of colonies×serial dilution factor×100 (as only a 10 μl sample was taken).

These cfu values were then converted into log₁₀ values and plotted graphically against time of sample removal.

At each time point, samples were assessed in triplicate; the final cfu/ml value was an average (mean) of the three readings.

### Example 1

### Activity Against P. gingivalis & S. mutans (MIC & MBC Assays)

The following experiments were conducted using P. gingivalis NCTC 11834 and S. mutans ATCC 25175 as the test organisms.

MIC and MBC assays, as described above, were carried out using the test compound t-butyl-p-hydroquinone (TBHQ), which was obtained from Sigma Aldrich, UK. The antimicrobial chlorhexidine (CHX), a compound found in many oral healthcare products and known for use against P. gingivalis and periodontal diseases, was used as a comparison; this compound was also obtained from Sigma Aldrich, UK.

The TBHQ was dissolved in ethanol and the CHX in distilled water. The TBHQ experiments were conducted in triplicate and the CHX ones in duplicate.

The results are shown in Tables 1 and 2 below, for TBHQ and CHX respectively.

TABLE 1
TBHQ

| | MIC | MBC | MIC/MBC |
|---|---|---|---|
| Test organism | (μg/ml) | (μg/ml) | ratio |
| P. gingivalis NCTC 11834 | 0.49 | 0.98 | 0.5 |
| S. mutans ATCC 25175 | 31.25 | 31.25 | 1 |

TABLE 2
CHX

| | MIC | MBC | MIC/MBC |
|---|---|---|---|
| Test organism | (μg/ml) | (μg/ml) | ratio |
| P. gingivalis NCTC 11834 | 0.98 | 0.98 | 1 |
| S. mutans ATCC 25175 | 0.625 | 1.25 | 0.5 |

The Table 1 data show that TBHQ is active against both micro-organisms, but in particular has an exceptionally high activity against the P. gingivalis strain tested, its MIC and MBC being an order of magnitude lower against this strain than against the S. mutans test organism.

The activity of TBHQ is moreover significantly greater against P. gingivalis than against other Gram-negative bacteria tested, for example Escherichia coli ATCC 25922 (MBC 500 μg/ml), Pseudomonas aeruginosa ATCC 27853 (MBC>1000 μg/ml), Acinetobacter baumanni ATCC 19606 (MBC 500 μg/ml) and Klebsiella pneumoniae ATCC 700603 (MBC 1000 μg/ml).

From a comparison of Tables 1 and 2, it can also be seen that TBHQ has activity comparable with that of the conventional antimicrobial chlorhexidine against the P. gingivalis test strain.

The surprisingly high activity of the quinone against P. gingivalis, and its selectivity for the organism, make it an ideal candidate for use in the treatment of periodontal diseases.

### Example 2

### Activity Against P. gingivalis & S. mutans (SCKAs)

The following experiments were conducted using P. gingivalis NCTC 11834 and S. mutans ATCC 25175 as the test organisms.

SCKAs, as described above, were carried out using TBHQ as the test compound. Chlorhexidine was used as a positive control and 2.5% ethanol as a negative control.

For the SCKA experiments, the TBHQ and chlorhexidine were both tested at concentrations of 0.2% w/v. The TBHQ was dissolved in ethanol and the chlorhexidine in distilled water. All the experiments were conducted in triplicate.

The results for P. gingivalis are shown in Table 3 and those for S. mutans in Table 4.

TABLE 3
P. gingivalis

| | Viable cell counts | | |
|---|---|---|---|
| | (Log₁₀ cfu/ml) | | |
| Treatment | 0 min | 0.5 min | 1 min |
| 0.2% (w/v) TBHQ | 9.32 | 6.50 | *3.00 |
| | (±0.1) | (±0.46) | (±0.0) |
| 0.2% (w/v) CHX | 9.29 | *3.00 | *3.00 |
| | (±0.11) | (±0.0) | (±0.0) |
| Control | 9.35 | 9.47 | 9.46 |
| | (±0.05) | (±0.01) | (±0.02) |

*3.00 = limit of detection for the assay

TABLE 4
S. mutans

| | Viable cell counts | | |
|---|---|---|---|
| | (Log₁₀ cfu/ml) | | |
| Treatment | 0 min | 0.5 min | 1 min |
| 0.2% (w/v) TBHQ | 7.64 | 7.56 | 7.36 |
| | (±0.07) | (±0.05) | (±0.02) |
| 0.2% (w/v) CHX | 8.05 | *3.00 | *3.00 |
| | (±0.15) | (±0.0) | (±0.0) |
| Control | 7.78 | 7.79 | 7.59 |
| | (±0.05) | (±0.05) | (±0.2) |

*3.00 = limit of detection for the assay

Again the quinone can be seen to be highly active against the P. gingivalis test strain, and far more active against the P. gingivalis strain than against the S. mutans one. Moreover its activity against the P. gingivalis strain tested is also comparable to that of the more conventionally used oral health care active chlorhexidine at identical concentrations after 1 minute's contact time.

### Example 3

### Activity Against P. gingivalis & S. mutans (Biofilm Disruption Assays)

The following experiments were conducted using P. gingivalis 381 and S. mutans ATCC 25175 as the test organisms.

Biofilm disruption assays, as described above, were carried out using TBHQ as the test compound and chlorhexidine as a positive control. The negative control was 5% ethanol.

For these experiments, the TBHQ and chlorhexidine were tested at concentrations of 0.2% w/v. The TBHQ was dissolved in ethanol and the chlorhexidine in distilled water. All the experiments were conducted in triplicate.

The results for P. gingivalis are shown in Table 5 and those for S. mutans in Table 6.

TABLE 5
P. gingivalis

| | Viable cell counts | | |
|---|---|---|---|
| | (Log₁₀ cfu/ml) | | |
| Treatment | 0 min | 2 min | 5 min |
| 0.2% (w/v) TBHQ | 7.74 | 6.52 | *2.00 |
| | (±0.1) | (±0.01) | (±0.0) |
| 0.2% (w/v) CHX | 7.74 | 6.08 | 6.29 |
| | (±0.1) | (±0.75) | (±0.39) |
| Control | 7.74 | 7.63 | 7.68 |
| | (±0.1) | (±0.14) | (±0.21) |

*2.00 = limit of detection for the assay

TABLE 6
S. mutans

| | Viable cell counts | | |
|---|---|---|---|
| | (Log₁₀ cfu/ml) | | |
| Treatment | 0 min | 2 min | 5 min |
| 0.2% (w/v) TBHQ | 6.36 | 6.52 | 6.24 |
| | (±0.35) | (±0.36) | (±0.23) |
| 0.2% (w/v) CHX | 6.36 | 5.00 | 5.21 |
| | (±0.35) | (±0.77) | (±0.15) |
| Control | 6.36 | 6.24 | 6.51 |
| | (±0.35) | (±0.15) | (±0.06) |

FIG. 1 is a scanning electron microscopy (SEM) image of a P. gingivalis 381 biofilm formed on a hydroxyapatite disc after incubation in Wilkins-Chalgren broth (pH 7) for 96 hours under anaerobic conditions (×7000 magnification). FIG. 2 is a SEM image of a S. mutans ATCC 25175 biofilm formed on a hydroxyapatite disc after incubation in Brain-Heart Infusion broth supplemented with sucrose (5% w/w) for 48 hours in an atmosphere containing 5% CO₂ (×6000 magnification). These demonstrate successful biofilm formation using the test organisms.

The tabulated data demonstrate the activity of the test quinone against sessile bacteria (ie bacteria present in a biofilm, as observed in FIGS. 1 and 2 for the P. gingivalis and S. mutans test strains respectively), again in particular for the P. gingivalis test strain. They also show its activity to be significantly greater than that of the more conventionally used oral health care active chlorhexidine.

The results indicate the suitability of the quinone for the treatment of P. gingivalis infections in vivo. It may therefore be used to disrupt and/or suppress biofilm formation by the relevant bacteria, and/or to reduce, kill or at least inhibit bacterial colonies established within biofilms, for instance in the oral cavity. Its activity against P. gingivalis in biofilms, such as those involved in the formation of dental plaque, make the quinone particularly suitable for use either to reduce the risk of, or to treat an existing occurrence of, a periodontal disease.

### Example 4

### Activity Against P. gingivalis (Other Quinones)

This example used P. gingivalis NCTC 11834 as the test organism.

MIC and MBC assays, as described above, were carried out using the following alkyl- and halo-substituted benzo/hydroquinones as the test compounds:
thymoquinone (Sigma Aldrich, UK)
2,5-di-t-butyl-p-hydroquinone (Sigma Aldrich, UK)
thymohydroquinone (prepared by Syntopix, UK from thymoquinone sourced from Sigma Aldrich, UK)
2-t-butyl-p-benzoquinone (Sigma Aldrich, UK)
2-chloro-5-methyl-p-benzoquinone (Apin Chemicals, UK)
2-ethyl-p-hydroquinone (Apin Chemicals, UK)
2,3-difluoro-p-hydroquinone (Synthon Chemicals GmbH, Germany)
2,5-di-t-pentyl-p-hydroquinone (Sigma Aldrich, UK)
4-hexyl resorcinol (Sigma Aldrich, UK)
black cumin oil, containing thymoquinone (Codina Huile, France).

The solvents used were ethanol (for thymoquinone, 2,5-di-t-pentyl-p-hydroquinone and 4-hexyl resorcinol) and DMSO (for all other test compounds). The black cumin oil was used neat in all experiments.

The experiments were conducted in triplicate. The results are shown in Table 7 below; all are collated from a number of experiments.

TABLE 7

| | | | MIC/MBC |
|---|---|---|---|
| Compound | MIC | MBC | ratio |
| Thymoquinone | 0.49 | 0.98 | 0.5 |
| 2,5-di-t-butyl-p-hydroquinone | 15.6 | 15.6 | 1 |
| Thymohydroquinone | <0.49 | 0.98 | <0.5 |
| 2-t-butyl-p-benzoquinone | <0.49 | <0.49 | n/a |
| 2-chloro-5-methyl-p-benzoquinone | 0.98 | 1.95 | 0.5 |
| 2-ethyl-p-hydroquinone | 0.98 | 1.95 | 0.5 |
| 2,3-difluoro-p-hydroquinone | 3.9 | 7.8 | 0.5 |
| 2,5-di-t-pentyl-p-hydroquinone | 3.9 | 3.9 | 1 |
| 4-hexyl resorcinol | 3.9 | 7.8 | 0.5 |
| Black cumin oil | 0.5 | 0.5 | 1 |

The Table 7 data show that all the quinones tested demonstrated a high level of activity against P. gingivalis NCTC 11834. This in turn indicates the likely utility of such compounds in oral health care products, in particular for the treatment of periodontal diseases.

### Example 5

### Activity of Other Quinones Against S. mutans

Example 4 was repeated, but using S. mutans ATCC 25175 as the test organism.

The results are shown in Table 8 below; all are collated from a number of experiments.

TABLE 8

| | | | MIC/MBC |
|---|---|---|---|
| Compound | MIC | MBC | ratio |
| Thymoquinone | 3.9 | >250 | <0.0156 |
| 2,5-di-t-butyl-p-hydroquinone | 250 | >250 | <0.5 |
| Thymohydroquinone | 1.95 | 250 | 0.008 |
| 2-t-butyl-p-benzoquinone | 31.25 | 62.5 | 0.5 |
| 2-chloro-5-methyl-p-benzoquinone | 62.5 | 125 | 0.5 |
| 2-ethyl-p-hydroquinone | 31.25 | 125 | 0.25 |
| 2,3-difluoro-p-hydroquinone | 125 | 250 | 0.5 |
| 2,5-di-t-pentyl-p-hydroquinone | 125 | 250 | 0.5 |
| 4-hexyl resorcinol | 31.25 | 125 | 0.25 |
| Black cumin oil | 390 | 12500 | 0.03 |

The Table 8 data show that although many of the quinones tested were active against S. mutans ATCC 25175, in all cases the activity was lower than against P. gingivalis NCTC 11834. Thus, like TBHQ, these quinones appear to exhibit selectivity for the P. gingivalis strain.

### Example 6

### Topical Oral Health Care Formulations

Examples 1 to 5 above indicate the utility of an alkyl- or halo-substituted benzo/hydroquinone in treating infections caused by P. gingivalis, in particular infections within the oral cavity such as for example plaque formation, gingivitis or periodontitis. Such active agents may also be used for general oral health care, for example for the creation and/or maintenance of fresh-smelling breath. They may also be used to treat more systemic conditions associated with P. gingivalis infections, for example cardiovascular diseases.

A topical formulation for use in this way, against P. gingivalis and in particular to suppress and/or disrupt biofilm formation by P. gingivalis, may be prepared by formulating an alkyl- or halo-substituted benzo/hydroquinone, in particular an alkyl-substituted benzo/hydroquinone such as TBHQ, in a suitable fluid vehicle, optionally together with conventional additives as described above.

The formulation may be prepared and administered using known techniques. For topical application it may for example take the form of a paste, cream, gel, lozenge, buccal patch, spray, mouthwash or dentifrice, or it may be carried in or on a dental fibre or tape or a denture. It may contain additives which target the active ingredient(s) to a particular site within the oral cavity such as the gums or teeth or in particular the sub-gingival regions, and/or which otherwise control the release of the active(s) at the relevant site.

### REFERENCES

1. Overman P R. Biofilm: a new view of plaque. J. Contemporary Dent Pract 2000; 1(3): 1-8.
2. Holt S C, Ebersole J L. Porphyromonas gingivalis, Treponema denticola and Tannerella forsythia: the ‘red complex’, a prototype polybacterial pathogenic consortium in periodontitis. Periodontology 2000. 2005; 38: 72-122.
3. Teles R P, Haffajee A D, Socransky S S. Microbiological goals of periodontal therapy. Periodontology 2000. 2006; 42: 180-218.
4. Lamont R J, Jenkinson H F. Life below the gum line: pathogenic mechanisms of Porphyromonas gingivalis. Microbiol Mol Biol Rev 1998; 62(4): 1244-63.
5. Oliver R C, Brown L J, Loe H. Periodontal diseases in the United States population. J Periodontol 1998; 69(2): 269-78.
6. Albandar J M, Brunelle J A, Kingman A. Destructive periodontal disease in adults 300 years of age and older in the United States, 1988-94. J Periodontol 1999; 70(1): 13-29.
7. Papapanou P N. Epidemiology of periodontal diseases: an update. J Int Acad Periodontol 1999; 1(4): 110-6.
8. Brading M G, Marsh P D. The oral environment: the challenge for antimicrobials in oral care products. Int Dent J. 2003; 53(6 Suppl 1): 353-62.
9. Bosshardt D D, Lang N P. The junctional epithelium: from health to disease. J Dent Res. 2005 January; 84(1): 9-20.
10. Vrotos J A, Vrahopoulos T P. Effects of systemic diseases on the periodontium. Curr Opin Periodontol 1996; 3: 19-26.
11. Kuo L C, Polson A M, Kang T. Associations between periodontal diseases and systemic diseases: A review of the inter-relationships and interactions with diabetes, respiratory diseases, cardiovascular diseases and osteoporosis. Public Health 2007; 122: 417-33.

1-32. (canceled)

33. A method of treatment of a human or animal patient suffering from or at risk of suffering from a periodontal disease, or from a condition which is caused by, transmitted by or exacerbated by P. gingivalis (in particular by biofilm formation by P. gingivalis), the method involving topically administering to the patient a therapeutically or prophylactically effective amount of an alkyl-substituted benzoquinone, hydroquinone or mixture thereof, selected from 2-t-butyl-p-hydroquinone (TBHQ), 2-t-butyl-p-benzoquinone, thymoquinone, thymohydroquinone, and mixtures thereof.

34. A method according to claim 33, wherein the disease or condition is selected from dental plaque-induced gingival diseases; chronic (previously adult) periodontitis; aggressive periodontitis (formerly early-onset, prepubertal, juvenile or rapidly progressive periodontitis); necrotising periodontal diseases; abcesses of the periodontium; and post-operative bacterial infections which are caused by P. gingivalis.

35. A method according to claim 33, wherein the disease or condition is a bacterial infection of a wound or a lesion within the oral cavity.

36. A method according to claim 33, wherein the condition is subgingival plaque formation.

37. A method according to claim 33, wherein the benzo/hydroquinone or mixture is a mixture of an alkyl-substituted benzoquinone and its corresponding hydroquinone.

38. A method according to claim 33, wherein the benzo/hydroquinone or mixture is selected from 2-t-butyl-p-hydroquinone (TBHQ), 2-t-butyl-p-benzoquinone (TBBQ) and mixtures thereof.

39. A method according to claim 33, wherein the benzo/hydroquinone or mixture is used in a formulation which is in the form of a cream, paste, gel, lotion, foam, ointment, varnish or other viscous or semi-viscous fluid, or in the form of a spray, dropping fluid, aerosol or mouthwash.

40. A method according to claim 33, wherein the benzo/hydroquinone or mixture is used in a toothpaste, mouthwash, dentifrice, lozenge or buccal patch or is carried in or on a dental fibre or tape, an implant (including a chip for insertion into a periodontal pocket; a dental filling, bridge or cap; and a denture), a chewing gum, a tablet or a chewable capsule.

41. A method according to claim 33, wherein the benzo/hydroquinone or mixture is used in a formulation which additionally contains a surfactant.

42. A method according to claim 33, wherein the benzo/hydroquinone or mixture is used in a formulation which contains one or more additional agents selected from abrasives, polishing agents, bleaching agents, tooth whitening agents, surface active agents, detergents, foaming agents, sources of fluoride ions or fluorine-containing ions, zinc salts, non-cariogenic sweeteners, flavourings, menthol, desensitising agents, anti-tartar/sequestering agents, anti-calculus agents, sodium bicarbonate, anionic polycarboxylates, enzymes, humectants, thickeners, binders, pH regulating buffers, emulsifiers, solubilising agents, preservatives, colours/dyes, plant extracts, anti-plaque agents, additional antimicrobial agents and mixtures thereof.

43. A method for controlling the growth of P. gingivalis or of a biofilm produced by P. gingivalis, the method comprising applying, to an area or surface which is infected or suspected to be infected or capable of becoming infected with the organism, an alkyl-substituted benzoquinone, hydroquinone or mixture thereof selected from 2-t-butyl-p-hydroquinone (TBHQ), 2-t-butyl-p-benzoquinone, thymoquinone, thymohydroquinone, and mixtures thereof.

44. A method according to claim 43, wherein the area or surface is a non-living area or surface.

45. A method for disrupting and/or suppressing biofilm formation by P. gingivalis, the method involving the use of an alkyl-substituted benzoquinone, hydroquinone or mixture thereof, selected from 2-t-butyl-p-hydroquinone (TBHQ), 2-t-butyl-p-benzoquinone, thymoquinone, thymohydroquinone, and mixtures thereof.

46. A method for inhibiting, at least partially, the ability of P. gingivalis to invade junctional epithelia in the mouth, and/or to attach to or be internalised by non-keratinised epidermal cells in the mouth, the method involving the use of an alkyl-substituted benzoquinone, hydroquinone or mixture thereof, selected from 2-t-butyl-p-hydroquinone (TBHQ), 2-t-butyl-p-benzoquinone, thymoquinone, thymohydroquinone, and mixtures thereof.

47. Use of an alkyl-substituted benzoquinone, hydroquinone or mixture thereof, selected from 2-t-butyl-p-hydroquinone (TBHQ), 2-t-butyl-p-benzoquinone, thymoquinone, thymohydroquinone, and mixtures thereof, as an antibacterial agent against P. gingivalis.
